SEQUENCE LISTING (10 sequences)

SEQ ID NO: 1 - protein, 9 aa, Artificial sequence
  PEPTIDE 1..9: Human EBV antigenic peptide LMP1
YLLEMLWRL

SEQ ID NO: 2 - protein, 9 aa, Artificial sequence
  PEPTIDE 58..66: Human influenza antigenic peptide Flu
GILGFVFTL

SEQ ID NO: 3 - protein, 9 aa, Artificial Sequence
  PEPTIDE 91..99: amino acid sequence of antigenic peptide LLO91-99
GYKDGNEYI

SEQ ID NO: 4 - protein, 9 aa, Artificial sequence
  PEPTIDE 217..225: amino acid sequence of antigenic peptide p60217-225
KYGVSVQDI

SEQ ID NO: 5 - protein, 9 aa, Artificial sequence
  PEPTIDE 449..457: amino acid sequence of antigenic peptide p60449
IYVGNGQMI

SEQ ID NO: 6 - protein, 9 aa, Artificial sequence
  PEPTIDE 84..92: amino acid sequence of antigenic peptide Mpl84
GYLTDNDEI

SEQ ID NO: 7 - protein, 9 aa, Artificial sequence
  PEPTIDE 206..214: amino acid sequence of antigenic peptide GAD206
TYEIAPVFV

SEQ ID NO: 8 - protein, 9 aa, Artificial sequence
  PEPTIDE 546..554: amino acid sequence of antigenic peptide GAD546
SYQPLGDKV

SEQ ID NO: 9 - protein, 9 aa, Artificial sequence
  PEPTIDE: amino acid sequence of antigenic peptide NRP
KYNKANWFL

SEQ ID NO: 10 - protein, 9 aa, Artificial sequence
  PEPTIDE 91..99: amino acid sequence of antigenic peptide LLO91
LYLVCGERG

(19) United States
(12) Patent Application Publication
(10) Pub. No.: US 2003/0228258 A1
(43) Pub. Date: Dec. 11, 2003

(54) Suicide tetramers and uses thereof

(76) Inventors: David Scheinberg, New York, NY (US); Michael McDevitt, Bronx, NY (US); Rui-Rong Yuan, Fort Lee, NJ (US)

Correspondence Address: Benjamin Aaron Adler, ADLER & ASSOCIATES, 8011 Candle Lane, Houston, TX, 77071, US

(21) Appl. No.: 10/448,647

(22) Filed: May 30, 2003

Related U.S. Application Data
(60) Provisional application No. 60/384,581, filed on May 30, 2002.

(51) Int. Cl. A61K 51/00; C07K 14/74

(52) U.S. Cl. 424/1.69; 530/350

(57) ABSTRACT

The present invention provides a cytotoxic MHC I conjugate comprising a cytotoxic moiety, biotinylated MHC I monomers which each comprise an antigenic peptide and streptavidin, bound to the cytotoxic moiety or to a biotinylated cytotoxic moiety and to the biotinylated MHC I monomers. Alternative constructs comprising a cytotoxic moiety and biotinylated MHC I monomers where each monomer comprises an antibody fragment also are provided. The cytotoxic moiety may comprise an 225Ac radionuclide or other cytotoxin. Further provided are methods of killing CD8⁺T cell clonal populations.

### CROSS-REFERENCE TO RELATED APPLICATIONS

This non-provisional application claims benefit of provisional U.S. Serial No. 60/384,581, filed May 30, 2003, now abandoned.

### BACKGROUND OF THE INVENTION

1. Field of the Invention

This invention relates generally to the fields of immunology and radioimmunotherapy. More specifically, this invention relates to cytotoxic MHC I conjugates and uses thereof.

2. Description of the Related Art

Immune recognition by CD8⁺ T cells is determined by binding of αβ T cell receptors (TCR) to target cell antigen-derived peptides displayed in the target's major histocompatibility complex (MHC) class I molecule (1-5). These antigenic peptides can be non-native peptide fragments derived from foreign viral or bacterial proteins or derived from normal or mutated self proteins (3, 6-7).

The analysis of phenotypic and functional diversity in CD8⁺ T cells has been greatly enhanced by recently developed “tetramer” methodology. Soluble multimeric forms of peptide-MHC class I complexes or “tetramers” can bind stably to the T cell receptors on a given specific CD8 T cell clone (8-10). Such specific tetramers, which are fluorescently tagged, have been used to identify or isolate MHC class I-restricted and peptide-specific T cells from peripheral blood and other tissues (11-13). The specific T cell clone recognized can be quantitated by flow cytometry utilizing the tetramers tagged with fluorescent complexes.

High linear-energy-transfer (LET) alpha particle-emitters are of unique interest as cytotoxic agents because the alpha particle does not need to be internalized to kill cells. Furthermore, the alpha particles are potent enough and of such short range to selectively kill individual cells from outside of the cell while situated on the cell surface with cytotoxic potency approaching one alpha particle per cell. Bi-213 and At-211 alpha emitting antibody constructs are in human cancer trials (14).

Actinum-225 (Ac-225), with a 10 day half-life, is an alpha emitting atomic nanogenerator that decays to three daughter atoms, including bismuth-213 (²¹³Bi, T_{1/2}=46 min), thereby yielding four net alpha particle emissions (15). Previous studies using monoclonal antibodies conjugated to Ac-225 (²²⁵Ac) as therapy for cancer in animal models have revealed that very small doses, i.e., nanocurie amounts, of ²²⁵Ac-antibody are capable of specific cancer cell killing without significant toxicity (16-17). The characteristics of the alpha generators suggest that they would be useful in arming tetramers to selectively kill their cognate T cells clones.

Autoimmune disorders affect up to 3-5% of the general population in Western countries and two thirds of the patients are female (1, 2). The pathogenesis of most autoimmune disease remains poorly understood but it is believed to involve multiple elements, including certain environmental factors including infections, genetic defects and inappropriate immune responses, which lead to self damage and/or dysfunction. Autoimmune organ damage can be mediated by the activation of T cells, B cells, or both. Currently used immunosuppressive drugs are non-specific.

Cytotoxic CD8⁺ T cells with specificity for immunogenic peptide/MHC class I complexes play a critical role in the pathogenesis of several human disorders, including autoimmune diseases such as type I diabetes, multiple sclerosis, graft versus host disease and transplant rejection. Immune-mediated diabetes (type 1, IMD) is an incurable disease that is increasing in incidence throughout the Western world (50). Type I diabetes results from chronic autoimmune destruction of pancreatic β cells by an immune process that involves both CD4 and CD8 T lymphocytes in genetically prone individuals and is strongly influenced by the environment.

If the pathogenic T cell epitopes in a well-characterized autoimmune disorder are identified, then these peptide antigens can be included in tetramer constructs that are conjugated to potent cytotoxic agents thereby rendering them capable of killing specific CTL clones. Because it is possible to label tetramers easily with FITC, they should similarly be labeled with chelated-isotopes thus arming them to kill the CTLs rather than simply identifying them. As no methods are available to kill specific T cells clones because other immunosuppresive drugs kill broadly, a clonal deletion method is advantageous.

The inventors have recognized a need in the art for effective methods of targeting peptide- and MHC class I-restricted CD8⁺ T cell clones using a radiolabeled or toxin-labeled construct. Specifically, the prior art is deficient in methods of making stable armed and lethal radio- or toxin-labeled tetramers to target and to kill specific CD8⁺ T cells. The present invention fulfills this long-standing need and desire in the art.

### SUMMARY OF THE INVENTION

The present invention is directed to a cytotoxic MHC I conjugate comprising a biotinylated cytotoxic moiety, biotinylated MHC I monomers where each monomer further comprises an antigenic peptide and streptavidin which is bound to said biotinylated cytotoxic moiety and to the biotinylated MHC I monomers. The cytotoxic moieties described herein may comprise a Ac radionuclide or be another cytotoxin.

The present invention also is directed to a cytotoxic MHC I conjugate comprising biotinylated MHC I monomers where each monomer further comprises an antigenic peptide, an alpha-particle-emitting radionuclide chelated to a bifunctional moiety which is bound to the antigenic peptide and streptavidin which is bound to the biotinylated MHC I monomers. The cytotoxic moieties described herein may comprise a ²²⁵Ac radionuclide.

The present invention is directed further to a cytotoxic MHC I conjugate comprising a cytotoxic moiety and biotinylated MHC I monomers, where each monomer comprises an antibody fragment, bound to the cytotoxic moiety. The cytotoxic moieties described herein may comprise a ²²⁵Ac radionuclide or be another cytotoxin.

The present invention is directed further still to a method of killing a CD8⁺ T cell clonal population comprising contacting the clonal T cells with an effective amount of the cytotoxic MHC I conjugates described herein. Additionally, a method of purging a CD8⁺ T cell clonal population from bone marrow for a bone marrow transplant is provided. The method comprises contacting the clonal T cells in the bone marrow ex vivo with an effective amount of the cytoxic MHC I conjugates described herein and transplanting the bone marrow purged of said clonal T cells into a bone marrow recipient.

The present invention is directed further still to a method of constructing a cytotoxic MHC I conjugate comprising adding streptavidin to bind an admixture comprising the biotinylated cytotoxic moiety and the biotinylated MHC I monomers, both described herein. Alternatively, a method of constructing a cytotoxic MHC I conjugate comprising adding streptavidin to bind an admixture which itself comprises a biotinylated bifunctional moiety or the biotinylated cytotoxin described herein and the biotinylated MHC I monomers described herein and chelating an alpha-particle emitting radionuclide to the bound biotinylated bifunctional moiety is provided. Another alternative method of constructing a cytotoxic MHC I conjugate comprising adding streptavidin to bind the biotinylated MHC I monomers described herein and linking the alpha-particle-emitting labeled bifunctional moiety to the antigenic peptide described herein is provided.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1B depict an example of an armed tetramer having biotinylated antigenic peptide/MHC I monomers and a biotinylated chelated Ac-225 moiety bound to streptavidin in a 3:1:1 ratio (FIG. 1A) or of an armed tetramer having biotinylated antigenic peptide/MHC I monomers bound to streptavidin in a 4:1 ratio with a chelated Ac-225 moiety linked to an antigenic peptide (FIG. 1B).

FIGS. 2A-2D show tetramer_{PE} staining of human negative control Flu-specific CD8⁺ T cells using Flu/HLA-A2 tetramer (FIG. 2A) and human LMP₁ peptide-specific CD8 T cells using EB virus LMP₁/HLA-A2 tetramer (FIG. 2B) and of mouse p60₂₁₇₋₂₂₅ specific CD8⁺ T cells using p60₂₁₇₋₂₂₅ tetramer (FIG. 2C) and stimulated mouse splenic CD8 T cells using LLO₉₁/H-2K^{d} tetramers (FIG. 2D). Analysis of binding specificity was by flow cytometry. Dot plots were gated on live CD8 T lymphocytes and show tetramerPE staining. The percentage of activated tetramer-positive CD8 T cells is shown in the upper right quadrant.

FIGS. 3A-3B show specific binding of ¹¹¹In-LMP tetramers to human (FIG. 3A) and of ¹¹¹In-labeled LLO₉₁-tetramers to mouse (FIG. 3B) peptide-specific CD8 T cell lines. FIG. 3A: ¹¹¹In-LMP tetramers were tested against LMP₁-specific CD8 T cells (●), negative control Flu-specific CD8 T cells (O). Control ¹¹¹In-Flu tetramer was tested against LMP₁ CD8 T cells (▴). FIG. 3B: ¹¹¹In-labeled LLO₉₁-tetramers was tested against mouse LLO₉₁ specific CD8 T cells (●) and control p60₂₁₇₋₂₂₅ specific CD8 T cells (O). Data represent the mean of 2 tests from a single representive experiment that was done 3 times.

FIGS. 4A-4B demonstrates CD8 T cell surface binding to and internalization of ¹¹¹In-labeled LMP₁tetramers with LMP₁-specific CD8 or control Flu-specific CD8 T cell lines at 0° C. (FIG. 4A) or at 37° C. (FIG. 4B). Data represent the mean of two tests in a single representive experiment done 2 times.

FIG. 5 demonstrates armed ²²⁵Ac-labeled tetramer-specific human and mouse CD8 T cell killing. Data represent the mean of three tests in a single representive experiment done 2 times. FIG. 5A: Dose dependent cell killing of LMP₁-CD8 T cells (♦) or control Flu-CD8 T cells (O) by ²²⁵Ac-LMP, tetramers. Exposure to ²²⁵Ac-DOTA alone (▪) or cold LMP₁-tetramers (Δ) were used as controls. FIG. 5B: Dose dependent cell killing LLO₉₁—CD8 T cells (●) or control p60₂₁₇₋₂₂₅ cells (▴) by ²²⁵Ac-LLO tetramers. Exposure to cold LLO-tetramers (O) were used as a controls.

FIG. 6 demonstrates that ²²⁵Ac-LLO₉₁ tetramers selectively kill mouse LLO₉-specific CD8 T cells within a mixture of T cells. ²²⁵Ac-LLO₉₁ tetramers selectively killed LLO₉₁—CD8 T cells in a mixed cell culture (▴) and in cultures of purified LLO₉₁-CD8 T cells alone (●). ²²⁵Ac-LLO₉₁ tetramers produced minimal cytotoxicity in control P₆₀217-CD8 T cells (▪) (p<0.0001). Data represent the mean of three tests in a single representive experiment done two times.

FIG. 7 demonstrates that ²²⁵Ac-LLO₉₁ tetramers reduce γ-IFN secretion in targeted LLO₉₁ CD8⁺ T cells. Bars represent corrected % of spots from each CD8 cell line either treated with ²²⁵Ac-LLO₉₁ tetramers or controls.

FIG. 8 is a flow chart of a CTL clonal deletion method using L. monocytogenes infection in a murine model.

### DETAILED DESCRIPTION OF THE INVENTION

In one embodiment of the present invention, there is provided a cytotoxic MHC I conjugate comprising a biotinylated cytotoxic moiety, biotinylated MHC I monomers where each monomer comprises an antigenic peptide and streptavidin bound to the biotinylated cytotoxic moiety and to the biotinylated MHC I monomers.

In all aspects of this embodiment the biotinylated cytotoxic moiety may be an alpha-particle-emitting radionuclide chelated to a biotinylated bifunctional moiety or other biotinylated cytotoxin. The alpha-emitting radionuclide may be actinium-225 or bismuth-213. The cytotoxin may be saporin, ricin, gelonin or calicheamicin. Examples of the bifunctional chelating moiety are 1,4,7,10-tetraazacyclodododecane-1,4,7,19-tetraacetic acid or diethylenetriaminepentaacetic acid. Further in all embodiments the MHC I monomers may be HLA-A2 or H-2K^{d}. The antigenic peptides in all aspects may have an amino acid sequence comprising one of SEQ ID NOS: 1-10.

In a particular aspect the conjugate is a tetramer comprising the biotinylated cytotoxic moiety and the biotinylated antigenic peptide/MHC I monomers bound to streptavidin in a 1:4 ratio. An example of the cytotoxic moiety is an ²²⁵Ac-labeled bifunctional moiety. The antigenic peptide may have an amino acid sequence comprising one of SEQ ID NOS: 1-10.

In a related embodiment the present invention provides a cytotoxic MHC I conjugate comprising a ²²⁵Ac-labeled biotinylated bifunctional moiety, biotinylated MHC I monomers where each monomer comprises an antigenic peptide attached thereto and streptavidin bound to the ²²⁵Ac-labeled biotinylated bifunctional moiety and the biotinylated MHC I monomers. The bifunctional moieties, the MHC I monomers, the antigenic peptide and the tetramer construct are as described supra.

In another embodiment of the present invention there is provided a cytotoxic MHC I conjugate comprising biotinylated MHC I monomers where the monomers further comprise an antigenic peptide; an alpha-particle-emitting radionuclide chelated to a bifunctional moiety where the bifunctional moiety is bound to the antigenic peptide; and streptavidin bound to the biotinylated MHC I monomers. In this embodiment the alpha particle-emitting radionuclide may be actinium-225, astatine-211 or bismuth-213. Furthermore in this embodiment the conjugate may be a tetramer whereby the streptavidin is bound to the biotinylated antigenic peptide/MHC I monomers in a 1:4 ratio. In all aspects of this embodiment the bifunctional moieties, the monomers and the antigenic peptides are as described supra.

In a related embodiment the present invention provides a cytotoxic MHC I conjugate comprising biotinylated MHC I monomers which further comprise an antigenic peptide; a ²²⁵Ac-labeled bifunctional moiety where the bifunctional moiety is bound to the antigenic peptide; and streptavidin bound to the biotinylated MHC I monomers. In all aspects of this embodiment the bifunctional moieties, the monomers and the antigenic peptides are as described supra.

In yet another embodiment of the present invention there is provided a cytotoxic MHC I conjugate comprising a cytotoxic moiety and an MHC I monomer comprising an antibody fragment where the monomer is bound to the cytotoxic moiety. The antibody fragment may be an IgG fragment. In all aspects of this embodiment the cytotoxic moieties may be an alpha-particle-emitting radionuclide chelated to a biotinylated bifunctional moiety or may be a cytotoxin. The radionuclide, the bifunctional moiety, the cytotoxin, and the MHC I monomer are as described supra.

In a related embodiment there is provided a cytotoxic MHC I conjugate comprising a ²²⁵Ac-labeled biotinylated bifunctional moiety and an MHC I monomer comprising an antibody fragment where the monomer is bound to the bifunctional moiety. The bifunctional moiety, the MHC I monomer and the antibody fragment are as described supra.

In yet another embodiment of the present invention there is provided method of killing a CD8⁺ T cell clonal population comprising contacting the clonal T cells with an effective amount of any of the cytotoxic MHC I conjugates described supra. In this embodiment the clonal T cells are contacted in vitro, in vivo or ex vivo.

Further to this embodiment killing the CD8+ T cell clonal population may selectively block a CD8+ T cell clone mediated disease process. Examples of a CD8+ T cell clone mediated disease are an autoimmune disease, an infection, graft versus host diseases or transplant rejection. In a related embodiment there is provided a method of purging a CD8⁺ T cell clonal population from bone marrow for a bone marrow transplant in an comprising contacting the clonal T cells in the bone marrow ex vivo with an effective amount of any of the cytoxic MHC I conjugates described supra and transplanting the bone marrow purged of said clonal T cells into a recipient.

In still another embodiment of the present invention there is provided a method of constructing a cytotoxic MHC I conjugate, comprising adding streptavidin to bind an admixture which comprises the biotinylated cytotoxic moiety and the biotinylated MHC I monomers both described herein. Alternatively, in a related embodiment the method may comprise adding streptavidin to bind an admixture which comprises a biotinylated bifunctional moiety or the biotinylated cytotoxin described herein and the biotinylated MHC I monomers described herein and chelating an alpha-particle emitting radionuclide to the bound biotinylated bifunctional moiety. The bifunctional moiety and the radionuclide may be as described supra.

In both of these related embodiments the admixture comprises the biotinylated cytotoxic agent or the biotinylated bifunctional moiety and the biotinylated MHC I monomers in a ratio of about 1:3. Also, strepavidin is added to the admixture in an amount up to a 1:4 ratio.

In another related embodiment the cytotoxic MHC I construct may be constructed by adding streptavidin to bind said biotinylated MHC I monomers and linking the alpha-particle-emitting labeled bifunctional moiety to the antigenic peptide. The alpha particle-emitting radionuclide may be actinium-225, astatine-211 or bismuth-213. The MHC I monomers, the antigenic peptides and the bifunctional moieties may be as described supra.

The following definitions are given for the purpose of facilitating understanding of the inventions disclosed herein. Any terms not specifically defined should be interpreted according to the common meaning of the term in the art.

As used herein, the term “suicide tetramer” or “armed tetramer” shall refer to multimeric protein based construct that is capable of specific binding to its cognate cell by use of its specific MHC binding site and capable of killing such cognate T cell as a consequence of the arming of the tetramer with an isotope or toxin.

Provided herein are techniques for making stable alpha particle-emitting labeled or cytotoxin-labeled antigenic peptide/MHC I conjugates. Since it takes several hours to prepare purified radiolabeled tetramers, the much longer 10 day half-life of actinium-225 may be a more advantageous alpha emitting isotope to use when constructing a radiolabeled tetramer than would other alpha particle emitters, e.g., At-211 and B 1-213, with shorter half-lives, although such alpha emittors are not precluded. MHC I tetramers are conjugated to the alpha emitting atomic nanogenerator actinium-225 (225Ac) or to a cytotoxin, such as a cytotoxin, to selectively target specific peptide- and MHC class I-restricted CD8⁺ T cell clones. Such an approach may allow selective ablation of pathogenic T cell clones in vitro, in vivo or ex vivo without disturbing broader immune function.

The antigenic peptide specific CD8⁺ T cell clones used herein may be, although not limited to, CD8⁺ human anti-EBV or anti-influenza T cells or mouse anti-Listeria or diabetogenic T cells. A MHC I monomer comprises a heavy chain and a light chain, e.g., β2-microglobulin. The monomers may be HLA-A2 or H-2K^{d} monomers. The antigenic peptides may comprise a sequence of about 8-12 amino acids to fit in the binding groove of the folded structure of the monomer. Examples of antigenic peptide sequences are shown in Tables 2 and 3.

It is contemplated that a cytotoxin such as saporin, ricin, gelonin or calicheamicin may be used in the present invention. As demonstrated herein, radiolabeled tetramers specifically bind to and kill targeted CD8⁺ human anti-EBV or mouse anti-Listeria T cells at low doses while leaving unharmed non-specific control CD8⁺ T cell populations. However, because the internalization is low and some cytotoxins, e.g., gelonin, require internalization to be toxic, use of these cytotoxins may be not as efficacious as using a radionuclide or isotope.

The present invention also encompasses dimeric MHC I constructs. Alternatively an antibody fragment, such as IgG fragment, e.g., the constant domain of the IgG, may be fused to or attached to an MHC I monomer to form the dimer. The dimeric construct is radiolabeled or conjugated to other cytotoxins and can specifically target T cell clonal populations.

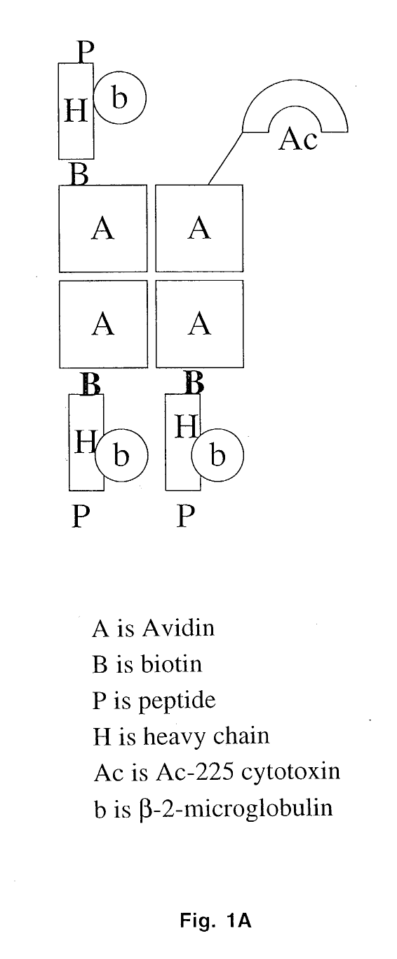

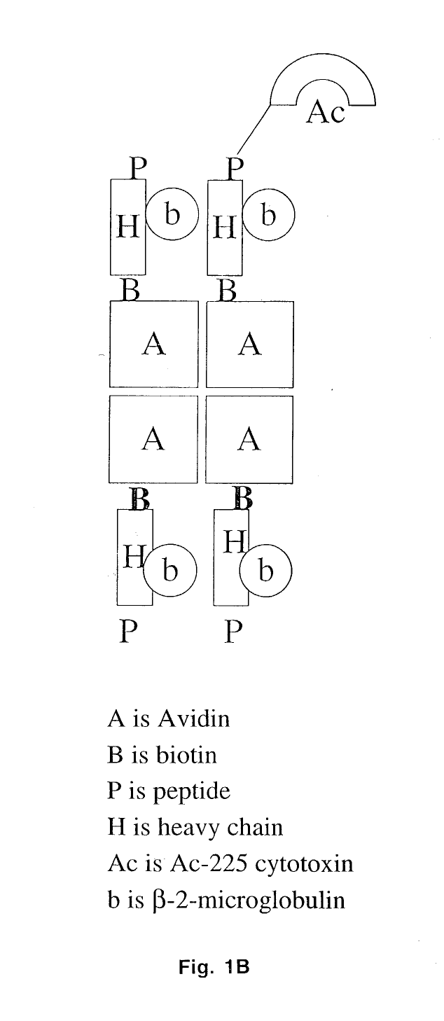

Although MHC I tetramers are known in the art, they are used solely to identify and to assay T cell clones and not for killing or deleting T cell clonal populations. The present invention provides a method of labeling them with an alpha particle emitting radionuclide or other cytotoxin. A biotinylated cytotoxic moiety or other biotinylated cytotoxin are admixed with biotinylated MHC I monomers attached to an antigenic peptide. The streptavidin binds both the biotinylated cytotoxic moiety and the biotinylated MHC I monomers comprising an antigenic peptide. Alternatively, a biotinylated bifunctional moiety or a biotinylated cytotoxin and the biotinylated monomers may be bound to the streptavidin to form the tetramer. If the tetramer comprises the biotinylated bifunctional moiety, a radionuclide is then chelated thereto to form the cytotoxic MHC I conjugate. To take advantage of the four binding sites of streptavidin has for biotin a tetramer comprising the cytotoxic moiety is constructed, preferably a 225Ac-antigenic peptide/MHC I tetramer (FIG. 1A).

It is further contemplated that the cytotoxic MHC I conjugate may comprise 4 antigenic peptide/MHC I monomers bound to streptavidin in a 4:1 ratio. The alpha particle-emitting bifunctional chelate is attached to the antigenic peptide via the bifunctional moiety without the necessity of biotinylating the moiety. The bifunctional moiety comprises a linker that covalently binds peptides (FIG. 1B). This is particularly useful for radionuclides, e.g., astatine that will not attach via biotin. This cytotoxic MHC I construct made be assembled by adding streptavidin to bind biotinylated MHC I monomers and linking the alpha-particle-emitting labeled bifunctional chelate to the antigenic peptide.

The cytotoxic MHC I constructs of the present invention provide a way to kill or induce apoptosis in specific CD8⁺ T cell populations. As such, these MHC I constructs may be useful in the treatment of or in the selective blocking of the CTL clone mediated disease process. Tissue destruction mediated by specific CTL clones is associated with several human autoimmune diseases such as diabetes, multiple sclerosis or vitiligo. Additionally, specific CTL clones may be involved in other pathogenic processes such as infection, graft versus host diseases and transplant rejection. Particularly, this strategy may be useful for ex vivo purging of minor antigen specific CTLs prior to bone marrow transplantation to prevent graft verses host diseases. Furthermore, the cytotoxic MHC I conjugates may be used as a research tool, e.g., to kill T cells for immunology research.

The cytotoxic MHC I conjugates presented herein may be included in a pharmaceutical composition for delivery to a mammal during a therapeutic strategy for CTL mediated diseases or processes. Compositions for and production of such pharmaceutical compositions are known in the art. Additionally, methods of generating and handling radionuclides for radioimmunotherapeutic processes are also known in the art and disclosed herein. One of skill in the art would be able to determine doses, specific activities and dosage regimens for the radionuclides and cytotoxins used and the diseases to be treated.

The following examples are given for the purpose of illustrating various embodiments of the invention and are not meant to limit the present invention in any fashion. Statistical analyses were performed using one-way ANOVA analysis of GraphPad Instat 3.0 (GraphPad Software).

### EXAMPLE 1

Animal Models

Animal studies are performed under protocols approved by the Institutional Animal Care and Use Committee (IACUC) of Memorial Sloan-Kettering Cancer Center. Animals are housed in microisolator cages under specific pathogen-free conditions. Female Balb/c mice (4-6 weeks old) and NOD mice are used.

### EXAMPLE 2

Human and Mouse CD8⁺ T Cell Lines

Human EB virus peptide and influenza virus peptide specific CD8⁺ T cell lines (LMP₁ or Flu₅₈₋₆₆) (18-20) and mouse Listeria monocytogenes specific CD8⁺ T cell clones specific for the Listeriolysin O (LLO)₉₁₋₉₉/H-2K^{d} or p6₂₁₇₋₂₂₅/H-2K^{d} (21-23) were used. Peripheral blood obtained from the same HLA-A2 healthy donor was used to establish both the human EBV virus peptide (LMP₁: YLLEMLWRL; SEQ ID NO: 1) and the influenza peptide (Flu: GILGFVFTL; SEQ ID NO: 2) specific cell lines. Briefly, purified mononuclear cells were obtained from peripheral blood by Ficoll-Hypaque separation. After NK cell and monocyte depletion, aliquots of the remaining lymphocyte population were stimulated in vitro by exposure to either irradiated autologous LMP₁- or Flu-peptide loaded EBV transformed B cells and cultured in special lymphocyte medium (AIM-V medium, GIBCO) containing 100 IU/ml IL-2 (BD Biosciences). After several weeks of stimulation, the enriched CD8⁺ T cell cultures subsequently were tested by LMP₁-tetramer or Flu-tetramer flow cytometry for binding specificity. Cells positive for tetramer binding were further stimulated and aliquots of the enriched human cells were used.

Murine Listeria peptide-specific CD8⁺ T cell lines were established from Balb/c splenocytes three weeks after immunization with a sublethal dose of Listeria (21-24). The LLO₉₁₋₉₉ peptide-(GYKDGNEYI; SEQ ID NO: 3) or p60₂₁₇₋₂₂₅ peptide-(KYGVSVQDI; SEQ ID NO: 4) specific CD8⁺ T cells were maintained in RPMI medium containing 0.16 μg/ml IL-7 (BD Biosciences) and 0.5 ng/ml IL-2 at 37° C. in 5% CO₂. Tetramer binding specificity of the CD8⁺ T cells was reconfirmed using tetramer flow cytometry before each experiment. Tetramer binding to CD8⁺ cell lines was stable over several weeks when cells were maintained in culture with periodic exposure to peptide pulsed APCs and fresh cytokines. This allowed us to utilize the same mouse cell line repeatedly for study.

### EXAMPLE 3

Peptide/MHC I Tetramer Constructs

Peptide/HLA-A2 tetramers or Peptide/H-2K^{d} tetramers were prepared as previously described (20,25) and provided by the MSKCC Tetramer Core Facility. Briefly, recombinant HLA A2 or H-2K^{d} and human β2 microglobulin produced in Escherichia coli were solubilized in urea and reacted with synthetic peptide antigens in a refolding buffer. The peptides used in this study were synthesized by ResGen Inc. (Huntsville, Ala.) and were >90% pure. Refolded peptide/MHC I complexes were purified and then biotinylated. Tetrameric peptide/MHC I complexes subsequently were produced by the stepwise addition of streptavidin-conjugated phycoerythrin (PE) to achieve a 1:4 molar ratio.

### EXAMPLE 4

Peptide/MHC I Gelonin Tetramer Constructs

Biotinylated gelonin was mixed with freshly prepared monomers in the presence of streptavidin tagged with FITC at a ratio of 1:3:1 in order to construc and assay armed immunotoxin tetramers. The product was further purified by size exlusion chromatography using 10 ml Econo-Pac 10 DG column (BioRad Lab, CA) with a PBS mobile phase. Both specific and non-specific tetramers were prepared in this manner for tetramer binding and cell killing experiments.

### EXAMPLE 5

Flow Cytometry

The binding specificity of individual tetramers was analyzed by flow cytometry using the previously characterized human and mouse specific CD8 T cell lines [20, 24]. Human LMP₁-specific CD8 T cells (1×10⁵ in 100 μl) were stained with FITC-anti-CD8 antibody (BD Biosciences) and PE-labeled LMP₁-tetramers at different concentrations at 4° C. for 60 min. Binding with control Flu/HLA A2 tetramers was employed to confirm the specificity of the LMP₁-tetramer staining. Flow cytometry using LLO₉₁₋₉₉/H-2K^{d} or p60₂₁₇₋₂₂₅/H-2K^{d} tetramers on mouse CD8 T cell lines was also conducted using identical conditions. To determine the stability of tetramer binding to TCRs, CD8 T cell lines were incubated with either specific or non-specific tetramers at 37° C. for 1, 4, 8 and 24 hrs, and binding was subsequently quantified by flow cytometry.

### EXAMPLE 6

IFN-γ ELISPOT Assay and ⁵¹Cr Release Assay for Cytotoxicity

The IFN-γ ELISPOT assay was performed in nitrocellulose-lined 96-well microplates (Millipore MAHA S45) using an IFN-γ ELISPOT kit. Plates were coated overnight with antibody to murine IFN-γ and washed six times. The LLO₉₁ CD8 T cells at 1×10⁶/ml or cells from a control p₆₀-217 CD8 T cell line at 1×10⁶/ml were incubated at 37° C. for 72 hr with ²²⁵Ac-LLO₉₁ tetramers at 5-10 nCi/ml. The responder LLO or p217 CD8⁺ T cells were then washed and added at 10⁵/well together with irradiated APC P815 cells and cognate peptides at 50 μg/mL and incubated for 20 h at 37° C. Wells containing CD8⁺ T cells and APC cells or non-specific control peptide served as negative controls.

The spots were counted using a stereomicroscope at a 40-fold magnification and an automated Elispot reader system (Carl Zeiss Vision, Germany) with KS Elispot 4.0 software. The final number of specific IFN-γ spots was obtained after subtracting the number of nonspecific IFN-γ spots produced in the control wells. All assays were performed in duplicate. The ⁵¹Cr release assay for determining cytotoxicity was performed as previously described (26-27). Target cells were labeled with ⁵¹Cr, coated with 10⁻⁶ M of either LLO₉₁₋₉₉ or p60₂₁₇₋₂₂₅ and incubated in the presence of enriched CD8 T cells at an E:T ratio of 100:1. After 5 h of incubation, CTL activity was calculated as the percentage specific ⁵¹Cr release from the targeted P815 cells using the equation: 100×[(experimental−spontaneous release)/(total−spontaneous release). Each assay was performed in triplicate.

### EXAMPLE 7

Preparation of ²²⁵Ac-DOTA-Biotin and ¹¹¹In-DTPA-Biotin Radionuclide ¹¹¹In was purchased from PerkinElmer life sciences (Billerica, Mass.) and ²²⁵Ac was obtained from the Oak Ridge national laboratory (Oak Ridge, Tenn.). The ²²⁵Ac nitrate residue was dissolved in 0.2 M Optima grade HCl (Fisher Scientific, PA) and biotinylated 1,4,7,10-tetraazacyclodododecane-1,4,7,19-tetraacetic acid (biotin-DOTA) was generously prepared by Dr. William Bornmann (MSKCC). Biotin-DOTA or biotinylated diethyenetriaminepentaacetic acid α, w-bis (DTPA, Sigma) was dissolved in metal-free water to yield a 10-20 mg/ml solution. The same procedure was used to label either biotin-DOTA (1 mg) with 1 mCi of ²²⁵Ac or biotin-DTPA (1 mg) with 2 mCi of ¹¹¹In.

Briefly, ²²⁵Ac was dissolved in 0.2 M HCl (5 μl to 20 μl) and added to a NUNC 1.8 ml reaction tube (Fisher Scientific, PA). One mg of biotin-DOTA solution (100 pi) was added along with 100 μl of 0.2 M HCl, 50 μl of 2M tetramethylammonium acetate and 15 μl of 150 g/L I-ascorbic acid (Aldrich Chemical Co. WI). The mixture (pH=4.5-5.0) was then heated to 60° C. for 30 minute and the reaction was terminated by adding 20 μl of 0.10 M EDTA (Aldrich Chemical Co.). The ¹¹¹In-DTPA biotin mixture was prepared without the heating step before termination.

To quantitate incorporation of ²²⁵Ac or ¹¹¹In radionuclide, 1 ml of Sepadex C-25 resin (Aldrich Chemical Co.) in 0.9% NaCl was packed into a column. A 2 μl aliquot of the radioactive reaction mixture was applied and the column was eluted with 3 ml of 0.9% NaCl. The column was eluted a second time to determine if all radioactivity had been removed. The column and washes were either counted immediately using a Squibb CRC-17 Radioisotope Calibrator to measure ¹¹¹In activity or counted 20 hours later to determine ²²⁵Ac activity levels. The activity contained in the eluate was considered to be the ¹¹¹In or ²²⁵Ac that was complexed to the chelant moiety.

Biotin reactivity in the radiolabeled component was assayed after application of the radioactive reaction mixture to an immobilized avidin column (Pierce, Ill.). The column was washed twice with 5 ml of 0.9% NaCl to remove unbound material and the column and washes were counted to determine the ¹¹¹In or the ²²⁵Ac activities using the same method previously described above. The % activity bound to the column was considered to be the ¹¹¹In or ²²⁵Ac that contained biotin-avidin binding reactivity.

### EXAMPLE 8

¹¹¹In Radiolabeled Tetramer Constructs

¹¹¹In has a relatively short half life of ˜3 days and it was selected to establish the optimal conditions for tetramer labeling. The freshly prepared ¹¹¹In-DTPA-biotin products were mixed with biotinylated monomers in the presence of streptavidin at a ratio of 1:3:1 in order to construct radiolabeled tetramers. The product was further purified by size exclusion chromatography using a 10 ml Econo-Pac 10DG column (BioRad lab, CA) with a PBS mobile phase. Both radiolabeled specific and non-specific tetramers were prepared in this fashion for in vitro studies. In addition, non-radiolabeled cold tetramers used for controls and blocking experiments were similarly prepared.

### EXAMPLE 9

Specific Binding and Internalization of ¹¹¹In Radiolabeled Tetramers

Different dilutions of radiolabeled tetramers in 5-8 μl PBS were added to 5-10×10⁶ tetramer specific CD8⁺ or control CD8⁺ T cells on ice. To determine the influence of incubation temperature on radiolabeled tetramer binding or tetramer internalization, cells were incubated either with radiolabeled tetramers on ice or at 37° C. for 1, 2, 4 hrs and overnight. The cells were then pelleted, washed twice with 1 ml ice cold PBS and subjected to scintillation counting to determine the amount of specific ¹¹¹In-tetramer binding.

To quantitate internalization of radiolabeled tetramers, the cell surface-bound radiolabeled tetramers were stripped from pelleted cells by exposure to 1 ml of 50 mM glycine/150 mM NaCl at pH 2.8 for 10-15 minutes at room temperature. The quantity of surface-bound and internalized radioactivity was determined by counting the samples separately. Both radiolabeled non-specific control tetramers and CTL cell lines bearing TCR of different peptide specificities served as controls for this assay. All assays were performed in duplicate.

### EXAMPLE 10

Specific Cell Killing by ²²⁵Ac Labeled Tetramers

The killing efficacy of suicide tetramers was quantitated using 1×10⁵ LMP₁ or LLO₉₁ specific CD8⁺ T cells in 96-well plates. Flu-specific or p60₂₁₇-specific CD8⁺ T cells served as negative controls. Serial dilutions of ²²⁵Ac-tetramers were added to the CD8⁺ T cells and non-specific cell killing was determined by adding only ²²⁵Ac-DOTA or only non-radiolabeled tetramers. In addition, some cells were incubated first with a 50-100 fold excess of non-radiolabeled specific tetramers before plating and subsequent addition of suicide tetramers to confirm specificity of cell killing by blockade. The cells were incubated for 48-96 h at 37° C. in 5% CO₂ and cell viability was subsequently determined by [³H] thymidine incorporation. Trypan blue testing was also used to determine cell viability in the CD8⁺ T cell lines. Each assay was performed in triplicate. In addition, the viable murine Listeria peptide-specific CD8⁺ T cells were washed 72 hrs post suicide tetramer treatment. Specific cytotoxicity and γ-IFN secretion were measured by ⁵¹Cr release and Elispot assays before exposure to suicide tetramers and compared to obtained baseline values.

In order to further demonstrate that ²²⁵Ac-suicide tetramer killing was selected to the appropriate TCR positive CTL cells, the ²²⁵Ac-LLO₉₁ tetramers were added to a mixed cell culture of LLO₉₁ tetramer positive CD8⁺ T cells and LLO tetramer negative, p60₂₁₇₋₂₂₅ specific CD8⁺ T cells. Serial dilutions of suicide ²²⁵Ac-LLO tetramers were added to the cell mixture containing 5×10⁴ LLO₉₁—CD8⁺ cells and 5×10⁴ p60₂₁₇₋₂₂₅ specific CD8⁺ cells. Cell viability was subsequently determined by Trypan blue staining, [³H]-thymidine incorporation after incubation at 37° C. in 5% CO₂ for 72 h. The remaining viable CD8⁺ T cells were washed and then restudied by tetramer flow cytometry to define and quantitate their target specificities. Each assay was performed in triplicate.

### EXAMPLE 11

High Specificity Selective Binding of Peptide Specific Tetramers to CD8⁺ T Cell Lines

The tetrameric structure assembled from peptide-MHC class I monomers is highly specific for its cognate antigen-specific CD8⁺ T cell clone (8). To establish the best conditions for constructing radiolabeled tetramers, non-radiolabeled tetramers first were prepared by stepwise addition of PE- or FITC conjugated streptavidin to purified biotinylated peptide/MHC class I monomers. The final product was purified by size exclusion chromatography and tetramer specific binding to cells was quantified using the human and mouse CD8⁺ T cell lines (1×10⁵ cells/sample).

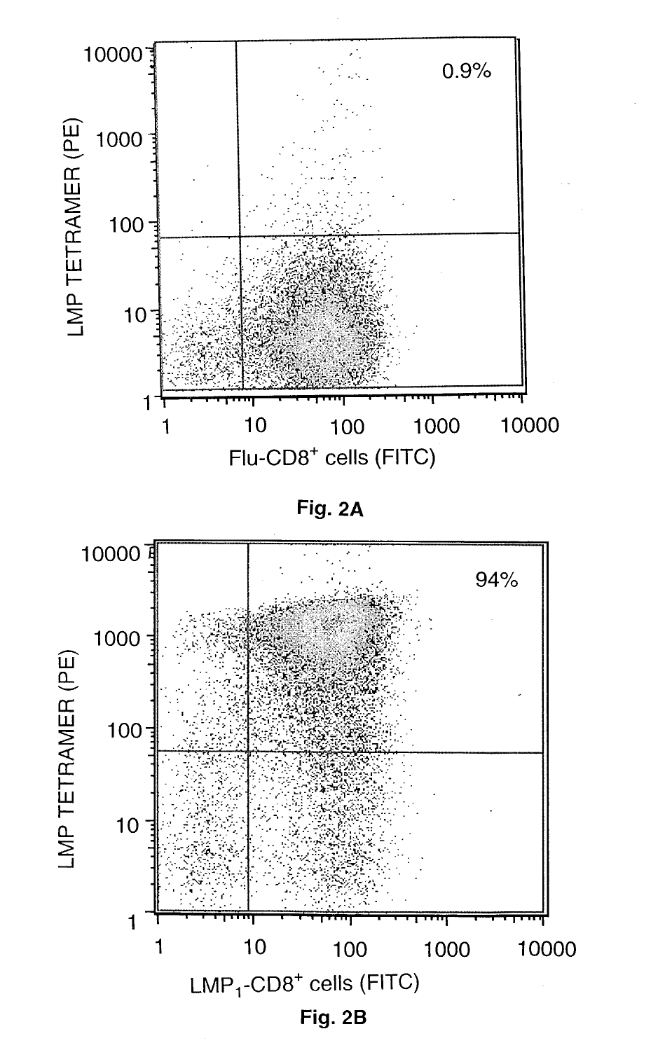

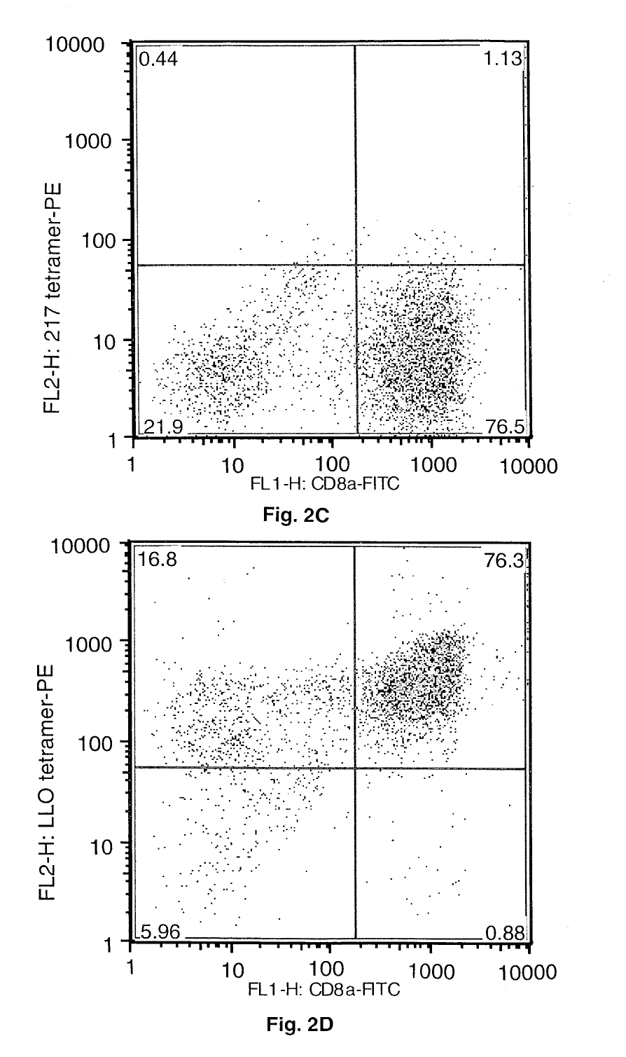

More than 90% of enriched human LMP₁ specific CD8⁺ T cells avidly bound the LMP₁/HLA-A2 tetramer, while only 1% of the control Flu-specific CD8⁺ T cells stained with the LMP₁ tetramer (FIGS. 2A-2B). LMP₁-specific cells were similarly negative for peptide Flu/HLA-A2 tetramer reactivity. To compare the relative binding affinity of different sized multimers, different fractions of the multimeric LMP₁ peptide/HLA-A2 reaction mixture were collected separately by size exclusion chromatography and incubated with LMP₁ specific CD8⁺ T cells. 95% of the cells were highly reactive with the tetramer, 89% with the trimer, 67% with the dimer and <30% cells stained with the monomer (data not shown).

Similar experiments were performed using mouse CD8⁺ T cell lines specific for L. monocytogenes peptides. 76% of enriched mouse splenic CD8⁺ T cells bound to the LLO₉₁ tetramer while only 1.3% of the control p60₂₁₇₋₂₂₅ specific CD8⁺ T cells stained with the LLO₉₁ tetramer (FIGS. 2C-2D). Similarly, 70% enriched murine for p60₂₁₇₋₂₂₅ CD8⁺ T cell line was stained positive for p60₂₁₇₋₂₂₅ tetramer and there was no cross reactivity between LLO₉₁ and p60₂₁₇₋₂₂₅ specific CD8 T cell lines, a finding consistent with previous reports (28). This confirms that these CD8 T cell receptors are peptide and MHC class I molecule specific.

To verify tetramer binding stability under more physiologic conditions, LMP₁-specific CD8 T cells were incubated with either specific or non-specific tetramers at 37° C. and binding was quantitated after different time intervals (1, 4, 8 and 24 hrs). Tetramer binding at 37° C. increased with prolonged incubation (8 hrs >4 hrs >1 hr) and was maximal after an 8 hr incubation as shown in Table 1.

TABLE 1
Stability of tetramer binding to CD8⁺ T cell receptors at 37° C. over time

Incubation Time (hours)

| CD8⁺ T cells/Tetramers | 1 | 2 | 8 | 24 |
|---|---|---|---|---|
| LMP₁/LMP₁ | 92% | 94% | 99% | 86% |
| Flu/LMP₁ | 0.9% | 1.2% | 1.5% | 1% |
| LLO₉₁/LLO₉₁ | 76% | 75% | 63% | |

T cell specificity was retained at 37° C. and no binding to the control Flu-specific CD8 T cell line was seen. These findings confirm that tetramers are capable of maintaining structural stability for several hours at 37° C., a feature that would be important for use in vivo. The progressive increase in CD8 T cell tetramer binding observed during the first 8 hours of incubation at 37° C. may reflect augmented TCR expression secondary to T cell activation. In addition, the slight decrease in tetramer binding observed after incubation for 24 hours may be a consequence of tetramer internalization as reported previously (30).

### EXAMPLE 12

Specific Binding of Radiolabeled Tetramers to CD8 T Cell Lines

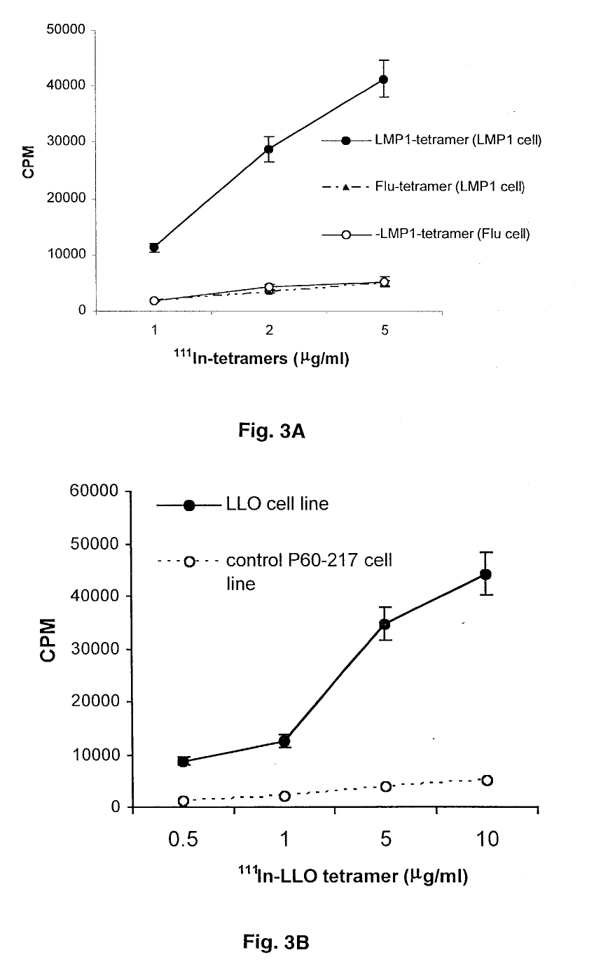

¹¹¹In, a pure gamma emitting isotope with a 3 day half life was used as a radiolabel to determine the efficacy of conjugating an alpha emitting radionuclide to peptide/MHC I multimers. Radiolabeled tetramers were assembled by adding one ¹¹¹In-DTPA-biotin and three biotinylated monomers for each streptavidin molecule since each molecule of streptavidin has four biotin binding sites. Highly purified ¹¹¹In-biotinylated DTPA (99%) was obtained and used for tetramer labeling. The final product was purified by separating the radiolabeled multimers from non-labeled small size products by passage through an Econo-PacIOG column. Non-radiolabeled fluorescent tetramers were tested for specific binding and served as an additional quality control for use when assembling radiolabeled tetramers. 1×10⁷ human LMP₁-specific or negative control Flu-specific CD8 cells were incubated with ¹¹¹In-LMP₁ tetramers at different concentrations for 30 min on ice to determine if they displayed specific binding (FIG. 3A). Specific binding of ¹¹¹In-LMP₁ tetramers to cells was measured after washing twice with PBS. ¹¹¹In-labeled LMP₁ tetramers exhibited dose dependent, specific binding to the LMP₁ CD8+clone. In contrast, there was little binding of ¹¹¹In-LMP₁ tetramers to the Flu-specific control CD8⁺ T cells. Additionally, ¹¹¹In-Flu tetramers showed little binding to LMP₁ CD8⁺ T cells even at very high concentrations. These results strongly indicate that the peptide specific tetramers could be successfully radiolabeled with maintenance of their binding specificity for the targeted CD8⁺ T cells. Furthermore, these observations were confirmed by a similar experiment testing ¹¹¹In-labeled LLO₉₁ tetramer binding against murine LLO₉₁— specific CD8⁺ T cells (FIG. 3B).

### EXAMPLE 13

Internalization of ¹¹¹In-Labeled Tetramers

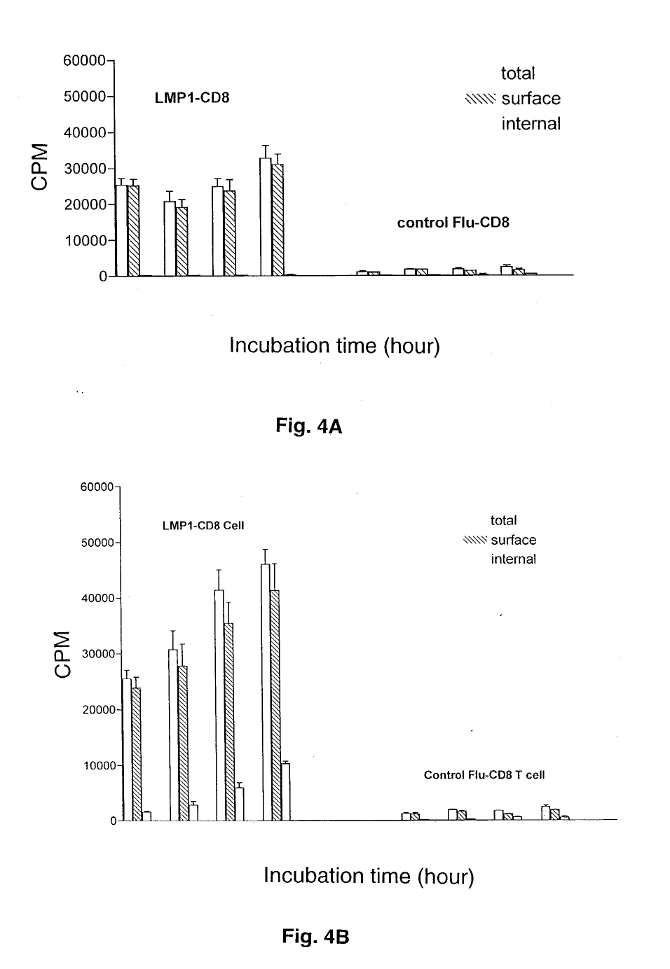

Efficacy of killing should be increased if the armed tetramers are internalized, though it is not a prerequisite for killing by alpha particles. To determine if the radiolabeled tetramers were internalized after binding to cell surface T cell receptors, the cells were incubated with ¹¹¹In-labeled tetramers and then their surface and internalized radiolabeled tetramers were measured. ¹¹¹In-LMP₁ tetramers (1 μg/ml) were added to LMP₁-specific CD8 or control Flu-specific CD8 T cell lines. Cells were then divided into two aliquots with one sample incubated on ice (FIG. 4A) while the other was reacted at 37° C. (FIG. 4B). Surface binding and internalization of ¹¹¹In-LMP₁ tetramers was measured at different time points.

At 0° C., only slightly increased cell surface binding of ¹¹¹In-LMP₁-tetamers to LMP₁-specific CD8 cells was observed with increasing time. Less than 2% internalization was observed at all time intervals tested. The control Flu-specific CD8 T cell line shows no binding to the ¹¹¹In-LMP₁ tetramer. Small amounts (6-8%) of ¹¹¹In-LMP₁ tetramers were internalized after a 1 hour incubation at 37° C. when tested on LMP₁ CD8⁺ T cells. Both cell surface binding and internalization of ¹¹¹In-LMP₁ tetramers to LMP₁-CD8 T cells progressively increase with prolonged incubation with a maximum of about 18%-22% of bound tetramers internalized after a 24 hr incubation. In contrast, there was little binding or tetramer internalization with the control Flu-specific CD8 T cell line (right).

### EXAMPLE 14

225Ac Labeled Tetramers Specifically Kill Targeted CD8⁺ T Cells

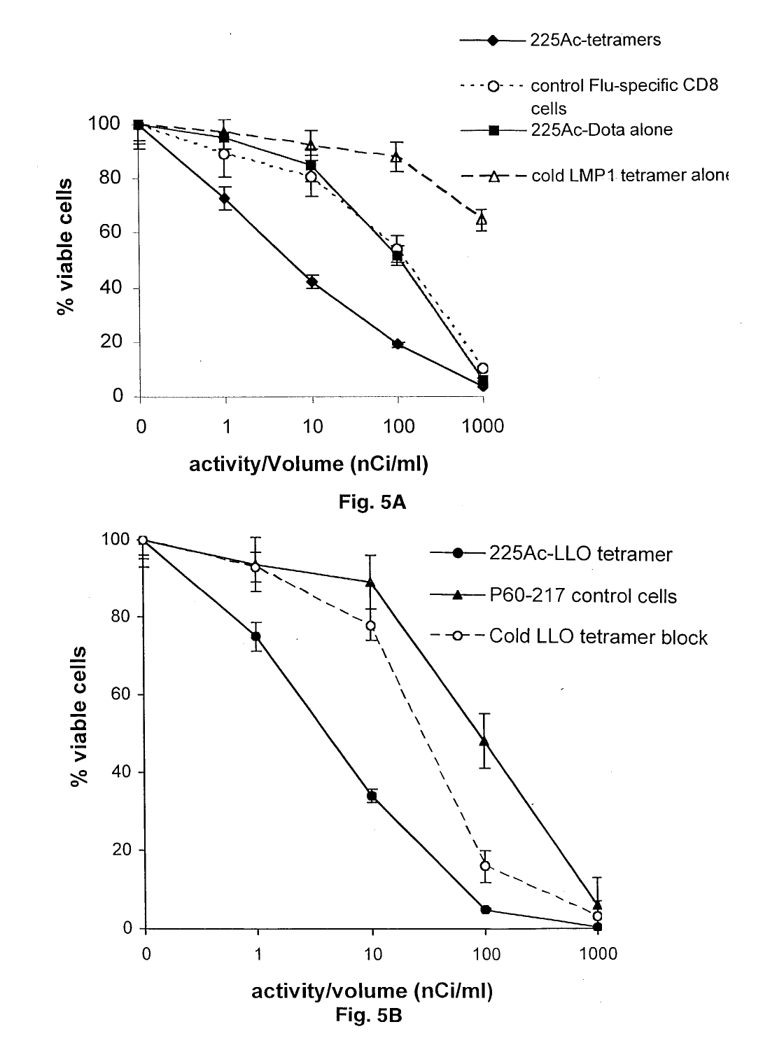

Based on the multimer labeling method for ¹¹¹In tracing, radiolabeled tetramers containing ²²⁵Ac generators for cell killing were constructed. Biotinylated DOTA was labeled with ²²⁵Ac at high yields (>96%). Tetramers were added to LMP1-CD8 T cells (1×10⁶/ml) or a control Flu-CD8 T cell line (1×10⁶/ml) and then incubated at 37° C. for 72 hr. The armed ²²⁵Ac-LMP₁ tetramers effectively killed the targeted LMP₁ CD8⁺ T cell clones at small doses (ED₅₀=5-8 nCi/ml) (FIG. 5A). In contrast, the armed ²²⁵Ac-LMP₁ tetramers exhibited minimal toxicity to control Flu-specific CD8⁺ T cells at similar low doses. Substantial non-specific cytotoxicity was induced at 50-100 fold higher doses (ED₅₀=200-300 nCi) of ²²⁵Ac alone (P<0.001) or cold tetramers. 1000-fold higher levels of cold LMP₁ tetramer alone were also capable of inducing mild cytotoxicity in targeted CD8⁺ T cells, but failed to show cytotoxic effects at relevant lower doses.

Similar high potency specific cell killing by suicide ²²⁵Ac-LLO₉₁ tetramers was demonstrated in the murine system. LLO₉₁ peptide specific CD8⁺ T cells were effectively killed after incubation with ²²⁵Ac-LLO₉₁ tetramers (ED50=4-8 nCi/ml). Fifty folds larger amounts of ²²⁵Ac were required to kill control p60₂₁₇₋₂₂₅ cells (ED₅₀=100-200 nCi/ml). Addition of cold LLO₉₁ tetramers (50 fold) incompletely blocked cell killing of ²²⁵Ac-LLO₉₁ tetramer (FIG. 5B).

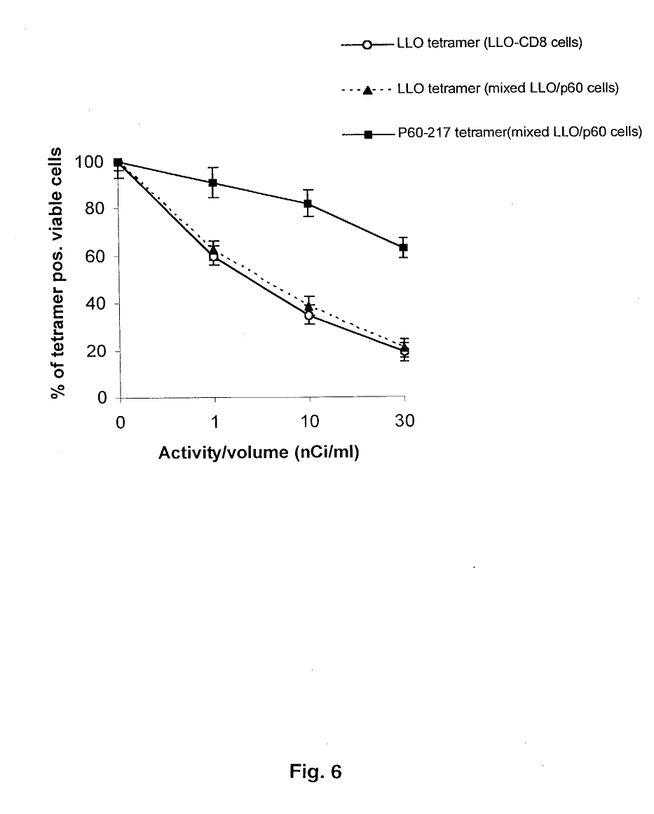

To corroborate that the killing of ²²⁵Ac-LLO₉₁ tetramers was restricted to LLO₉₁-peptide specific CD8⁺ T cells even within a mixture of possible target cells, ²²⁵Ac-LLO₉₁ tetramers at concentrations of 1-30 nCi/ml were added to 50:50 mixed cell cultures of LLO₉₁-specific CD8+ and P₆₀ 217-specific CD8⁺ T cells at 1×10⁶/ml (FIG. 6). After a 72 hour incubation with ²²⁵Ac-LLO₉₁ tetramers, significant cell killing was demonstrated by [³H] thymidine incorporation (ED₅₀=3-5 nCi/ml) and confirmed by counting dead/viable cells after Trypan blue staining (data not shown). When the remaining viable cells were analyzed by tetramer flow cytometry to define their tetramer specificities, there was a significant reduction in LLO₉₁ specific CD8⁺ T cells (p<0.001). In contrast, the P₆₀217 cell population in the mixed P₆₀217/LLO₉₁ cell culture showed only a slight reduction (<20%) even when exposed to higher quantities of ²²⁵Ac-LLO₉₁ tetramer (10 nCi/ml).

### EXAMPLE 15

γ-IFN Secretion After Treatment with ²²⁵Ac-Tetramers

The responder LLO₉₁ and p602]7 control CD8⁺ T cells were exposed to ²²⁵Ac-LLO₉₁ tetramers (10 nCi/ml) for 72 hr, washed, and then added to a 96-well microplate together with irradiated APC cells and either cognate or control peptide. Samples were performed in duplicate and the final number of specific IFN-γ spots was obtained after subtraction of nonspecific IFN-γ spots produced in control wells.

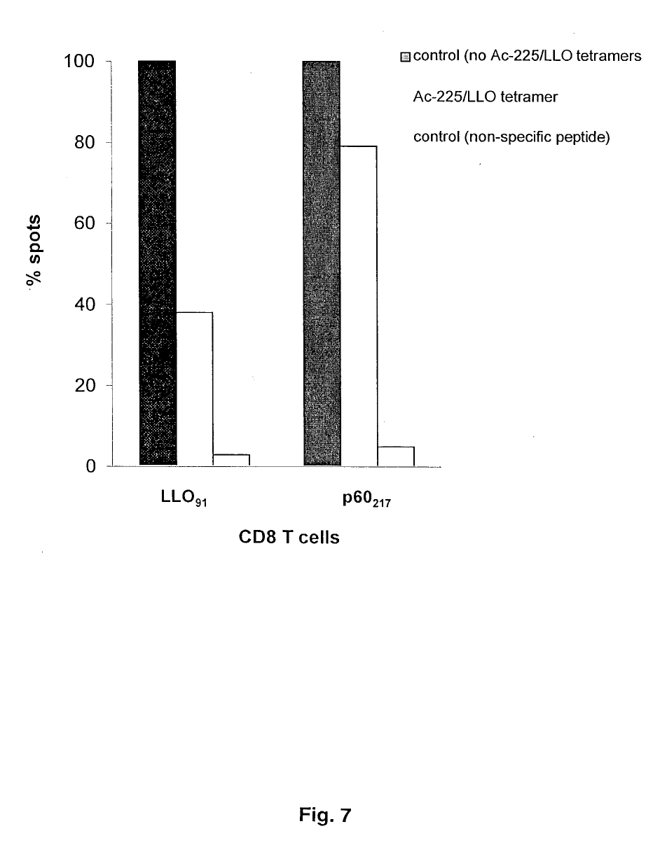

The level of γ-IFN secretion decreased significantly in suicide tetramer treated LLO₉₁ CD8⁺ T cells when compared to the non-treated control T cells (219±21 vs 83±14), while in contrast the control p60₂₁₇-specific CD8 T cells showed minimal reduction in γ-IFN secretion (133±7 vs 105±5) (FIG. 7). The cytotoxicity of suicide tetramer treated LLO₉₁ CD8⁺ T cells was even more significantly reduced and the function of the control p60₂₁₇ CTLs remained basically intact (data not shown). This demonstrates that ²²⁵Ac-LLO₉₁ tetramers can selectively delete LLO₉₁-tetramer positive CTLs with high specificity and induce little cytotoxicity within the other CD8⁺ T cell populations.

### EXAMPLE 16

Efficacy of Suicide Tetramers in an L. monocytogenes Infected Animal Model

This example quantifies Listeria-specific CTL clones from naive and immunized animal spleens and characterizes the efficacy of suicide tetramer exposure. Four major synthetic peptide antigens, as shown in Table 2, known to induce Listeria-specific CTL mediated immunity are prepared as described above. The peptide/H-2K^{d} tetramers are prepared as described above.

TABLE 2
L. monocytogenes peptide antigens for constructing
mouse tetramers

| Name of | Amino-acid | | |
|---|---|---|---|
| Peptides | Sequence | MHC class I | Score |
| LLO₉₁ | GYKDGNEYI | H-2K^{d} | 24 |
| | (SEQ ID NO:3) | | |
| ₚ60₂₁₇ | KYGVSVQDI | H-2K^{d} | 27 |
| | (SEQ ID NO:4) | | |
| ₚ60₄₄₉ | IYVGNGQMI | H-2K^{d} | 28 |
| | (SEQ ID NO:5) | | |
| Mpl₈₄ | GYLTDNDEI | H-2K^{d} | 26 |
| | (SEQ ID NO:6) | | |

Five Balb/c mice (5-6 weeks) are injected intravenously with a sublethal dose of 2,000 bacteria per mouse of wild type L. monocytogenes. Seven days after the primary infection, single-cell suspensions are prepared from mouse spleens and then incubated at 37° C. for 1 h in flasks to eliminate adherent cells before purification. CD8⁺ T cells are negatively selected by depletion of CD4⁺, MHC class II⁺, and CD11b⁺ cells using the MACS magnetic separation system. After incubation, cells are washed and are resuspended at 2×10⁸ cells/ml in PBS with 0.5% FBS. The mouse splenic CD8+cells from both infected and normal naïve mice are analyzed for their specific tetramer binding.

Specific cell killing is determined by incubating the established splenic T cell lines with each peptide specific suicide tetramer. T cells from normal BALB/c mouse spleens are used as the negative control. Splenic T cells (2×10⁴/well) are cultured in 96-well plates and serial dilutions of suicide [²²⁵Ac]tetramers are added to the wells. Non-specific radiolabeled tetramers also are used as controls. The plates are incubated for 24 h at 37° C. in 5% CO₂ and the cell viability is subsequently determined by [³H]thymidine incorporation or Trypan blue exclusion. The remaining viable cells are rechecked by tetramer flow cytometry to determine if the suicide tetramers have deleted only the specific CTL clonal population while leaving the remaining T cell population intact.

Establishing Specific CTL Lines In Vitro (47)

Balb/c mice are immunized by intravenous infection with a sublethal dose of 2,000 bacteria per mouse of L. monocytogenes. Single cell suspensions are prepared from spleens of mice 7-8 days post immunization with L. monocytogenes and RBCs lysed with ACK lysis buffer. Syngeneic splenocyte stimulators are prepared from naive mice by irradiation with 2600 rads and pulsed for 1 hr with selected peptide antigens at 50 μg/ml at 37° C. before being added to responder cells. These cells are incubated in 5% CO₂ at 37° C. for 72-96 hours in RPMI medium containing cytokines, IL-2 and IL-7, and re-stimulated weekly with peptides or peptide-coated stimulator cells. The tetramer binding specificity of the cells will be rechecked using tetramer flow cytometry before each experiment.

### EXAMPLE 17

Clonal Deletion of Antigen-Specific CD8⁺ T Cells to L. monocytogenes Infection In Vivo: Animal Model/Effectiveness of Suicidal Tetramers on Specific CTL Clonal Deletion Before and After Infection with L. monocytogenes

Radiolabeled tetramers complexed with Listeria-p60₂₁₇ peptide are diluted in 150 μl of sterile PBS and are injected intravenously or intraperitoneally into 10 Balb/c naive (5-6 weeks) mice. Administration of non-radiolabeled tetramer and use of untreated naive Balb/c mice serve as negative controls. Three days post treatment with suicide tetramers, the mice are injected intravenously with a sublethal dose (2,000 bacteria per mouse) of wild type L. monocytogenes. Seven days after the primary infection, CD8⁺ T cells are prepared as described above and the mouse splenic CD8⁺ cells are analyzed and quantified for their specific tetramer binding using all four epitope-specific tetramers LLO₉₁, p60₂₁₇, p60₄₄₉ and Mpl₈₄).

Elimination of the Recall Epitope-Specific CTL Response of CTLs After Specific Clonal Deletetion

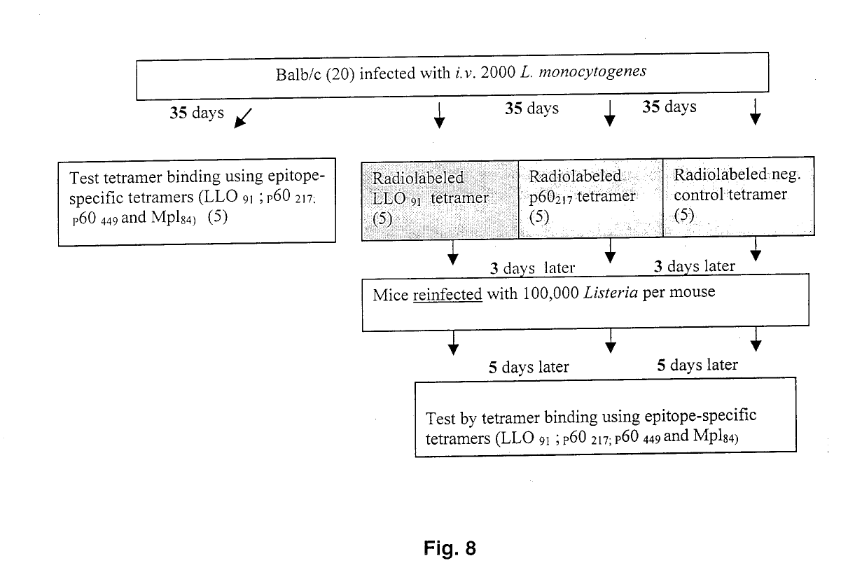

A total of 20 Balb/c mice are injected intravenously with 2,000 L. monocytogenes per mouse. Ten to fifteen days after the primary infection, single-cell suspensions are prepared from 5 mouse spleens and CD8⁺ T cells are prepared as described above and CTLs quantified for specific tetramer binding using the epitope-specific tetramers LLO₉₁, p60₂₁₇, p60₄₄₉ and Mpl₈₄). Groups of 5 mice are then treated with radiolabeled tetramers complexed with Listeria LLO₉₁, p60₂₁₇ or control peptide in 150 μl of sterile PBS injected intravenously or intraperitoneally. Three days post treatment with radiolabeled tetramers, the mice are challenged intravenously with 100,000 wild type L. monocytogenes per mouse. Five days after the re-infection, mouse splenic CD8⁺ T cells are prepared and quantified for their specific tetramer binding using all four epitope-specific tetramers LLO₉₁, _{P}60₂₁₇, _{P}⁶⁰₄₄₉ and Mpl₈₄) (FIG. 8). The data is analyzed by the Student's t test and a P value of less than 0.05 will be considered significant.

Pharmacokinetics and Toxicity of Radiolabeled L. monocytogenes Tetramers Injected Into Normal and Listeria-Immunized Balb/c Mice

The in vivo biodistribution of radiolabeled tetramers is determined in both normal naïve and Listeria-immunized Balb/c mice by testing organs after injection. The level of [¹¹¹In] that is tissue-associated is determined after intraperitoneal or intravenous injection of the tetramers diluted in 150 μl sterile PBS. Five mice from each group are sacrificed at 5 hrs, days 2 and 3 post injection, respectively, and blood, kidney, liver, intestine, heart, lungs, brains and bone marrow are removed and immediately counted with a scintillation counter.

Toxicity experiments are conducted using different doses of radiolabeled tetramers and 8-10 BALB/c mice per each dose group. They are monitored for viability, alteration in weight, hair loss and general condition twice a week. In addition, blood counts, blood chemistry values and histopathology are assessed at different time points. The data collected from toxicity experiments are analyzed using a DAX clinical analyzer (32).

Type 1 Diabetic Peptide Tetramers that Specifically Bind to Autoreactive CTL Cells in NOD Mice and the Specific Pathogenic CTL Clones

Synthetic peptide epitopes for constructing tetramers specific for diabetogenic CTL cell clones in NOD mice are shown in Table 3. Peptide LLO91-99 derived from L. monocytogenes is used as negative control (70).

TABLE 3
Diabetogenic peptide antigens for constructing
mouse tetramers

| Name of | Amino-acid | | |
|---|---|---|---|
| Peptides | Sequence | MHC class I | Score |
| GAD₂₀₆ | TYEIAPVFV | H-2K^{d} | 20 |
| | (SEQ ID NO:7) | | |
| GAD₅₄₆ | SYQPLGDKV | H-2K^{d} | 29 |
| | (SEQ ID NO:8) | | |
| NRP | KYNKANWFL | H-2K^{d} | 23 |
| | (SEQ ID NO:9) | | |
| G9 | LYLVCGERG | H-2K^{d} | 17 |
| | (SEQ ID NO:10) | | |
| LLO₉₁ (control) | GYKDGNEYI | H-2K^{d} | 24 |
| | (SEQ ID NO:3) | | |

The peptide/H-2K^{d} tetramers for animal study are prepared as described above. The amount of detectable autoreactive CTL population from both NOD and normal control mouse spleens is quantified using tetramer flow cytometry as described above.

Efficacy of the Radiolabeled Tetramers Injected into NOD Mice

Suicide tetramers are diluted in 150 μl of sterile PBS for the in vivo experiments and are injected intravenously or intraperitoneally. Groups of 10 treated and untreated mice are used for the initial in vivo study and normal non-diabetic BALB/c mice are used as negative controls. Administration of non-specific tetramers also are used as additional negative control. The treated and untreated mouse groups are monitored for development of diabetes by testing urine for glucose with a Chemistrip twice a week for up to one year. Mice with glucosuria are evaluated further by determining their blood glucose levels. Mice showing >250 mg/dl (>13.9 mM) glucose levels on two consecutive readings in a week are considered diabetic.

Pancreatic tissue from treated and control (5 mice per group) mice are collected at different intervals after suicide tetramer treatment and fixed in 10% buffered formalin or processed for immunohistochemistry. The pancreatic tissue is embedded in paraffin, sectioned, and stained with hematoxylin-eosin to assess the presence of mononuclear infiltrate in the pancreatic islets, i.e., insulitis. To assess the degree of pancreatic infiltration, sections are taken, and islets counted. To determine the percentage of peri-infiltrated or infiltrated islets, at least 10 islets are counted in at least two different fields. The data is analyzed by the Student's t test and a P value of less than 0.05 will be considered significant.

The following references are cited herein:

1. Monks, C. R., et al., Three-dimensional segregation of supramolecular activation clusters in T cells. Nature, 1998. 395(6697): p. 82-6.

2. Townsend, A. R., F. M. Gotch, and J. Davey, Cytotoxic T cells recognize fragments of the influenza nucleoprotein. Cell, 1985. 42(2): p.-457-67.

3. Goldrath, A. W. and M. J. Bevan, Selecting and maintaining a diverse T-cell repertoire. Nature, 1999. 402(6759): p. 255-62.

4. Davis, M. M. and P. J. Bjorkman, T-cell antigen receptor genes and T-cell recognition. Nature, 1988. 334(6181): p. 395-402.

5. Davis, M. M., et al., Ligand recognition by alpha beta T cell receptors. Annu Rev Immunol, 1998. 16: p. 523-44.

6. Watts, C., Capture and processing of exogenous antigens for presentation on MHC molecules. Annu Rev Immunol, 1997.15: p. 821-50.

7. Blattman, J. N., et al., Evolution of the T cell repertoire during primary, memory, and recall responses to viral infection. J Immunol, 2000. 165(11): p. 6081-90.

8. Altman, J. D., et al., Phenotypic analysis of antigen-specific T lymphocytes. Science, 1996. 274(5284): p. 94-6.

9. Constantin, C. M., et al., Major histocompatibility complex (MHC) tetramer technology: an evaluation. Biol Res Nurs, 2002. 4(2): p. 115-27.

10. Ogg, G. S. and A. J. McMichael, HLA-peptide tetrameric complexes. Curr Opin Immunol, 1998. 10(4): p. 393-6.

11. Dunbar, P. R., et al., Direct isolation, phenotyping and cloning of low-frequency antigen-specific cytotoxic T lymphocytes from peripheral blood. Curr Biol, 1998. 8(7): p. 413-6.

12. Ogg, G. S., et al., High frequency of skin-homing melanocyte-specific cytotoxic T lymphocytes in autoimmune vitiligo. J Exp Med, 1998. 188(6): p. 1203-8.

13. McDermott, A. B., et al., A simple and rapid magnetic bead separation technique for the isolation of tetramer-positive virus-specific CD8 T cells. Aids, 2001. 15(6): p. 810-2.

14. Sgouros, G., et al., Pharmacokinetics and dosimetry of an alpha-particle emitter labeled antibody: 213Bi-HuM195 (anti-CD33) in patients with leukemia. J Nucl Med, 1999. 40(11): p. 1935-46.

15. Ma, D., et al., Breakthrough of 225Ac and its radionuclide daughters from an 225Ac/213Bi generator: development of new methods, quantitative characterization, and implications for clinical use. Appl Radiat Isot, 2001. 55(5): p. 667-78.

16. McDevitt, M. R., et al., Tumor therapy with targeted atomic nanogenerators. Science, 2001. 294(5546): p. 1537-40.

17. McDevitt, M. R., et al., An alpha-particle emitting antibody ([213Bi]J591) for radioimmunotherapy of prostate cancer. Cancer Res, 2000. 60(21): p. 6095-100.

18. Khanna, R., et al., Identification of cytotoxic T cell epitopes within Epstein-Barr virus (EBV) oncogene latent membrane protein 1 (LMP1): evidence for HLA A2 supertype-restricted immune recognition of EBV-infected cells by LMP 1-specific cytotoxic T lymphocytes. Eur J Immunol, 1998. 28(2): p. 451-8.

19. Bednarek, M. A., et al., The minimum peptide epitope from the influenza virus matrix protein. Extra and intracellular loading of HLA-A2. J Immunol, 1991. 147(12): p. 4047-53.

20. Kochne, G., et al., Quantitation, selection, and functional characterization of Epstein-Barr virus-specific and alloreactive T cells detected by intracellular interferon-gamma production and growth of cytotoxic precursors. Blood, 2002. 99(5): p. 1730-40.

21. Villanueva, M. S., et al., Efficiency of MHC class I antigen processing: a quantitative analysis. Immunity, 1994. 1(6): p. 479-89.

22. Villanueva, M. S., A. J. Sijts, and E. G. Pamer, Listeriolysin is processed efficiently into an MHC class I-associated epitope in Listeria monocytogenes-infected cells. J Immunol, 1995. 155(11): p. 5227-33.

23. Busch, D. H. and E. G. Pamer, MHC class I/peptide stability: implications for immunodominance, in vitro proliferation, and diversity of responding CTL. J Immunol, 1998. 160(9): p. 4441-8.

24. Lauvau, G., et al., Priming of memory but not effector CD8 T cells by a killed bacterial vaccine. Science, 2001. 294(5547): p. 1735-9.

25. Busch, D. H., et al., Coordinate regulation of complex T cell populations responding to bacterial infection. Immunity, 1998. 8(3): p. 353-62.

26. Pamer, E. G., Direct sequence. identification and kinetic analysis of an MHC class I-restricted Listeria monocytogenes CTL epitope. J Immunol, 1994. 152(2): p. 686-94.

27. Murali-Krishna, K., et al., Counting antigen-specific CD8 T cells: a reevaluation of bystander activation during viral infection. Immunity, 1998. 8(2): p. 177-87.

28. Busch, D. H., K. M. Kerksiek, and E. G. Pamer, Differing roles of inflammation and antigen in T cell proliferation and memory generation. J Immunol, 2000. 164(8): p. 4063-70.

29. Maile, R., et al., Antigen-specific modulation of an immune response by in vivo administration of soluble MHC class I tetramers. J Immunol, 2001. 167(7): p. 3708-14.

30. Whelan, J. A., et al., Specificity of CTL interactions with peptide-MHC class I tetrameric complexes is temperature dependent. J Immunol, 1999. 163(8): p. 4342-8.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A cytotoxic MHC I conjugate, comprising:
a biotinylated cytotoxic moiety;
biotinylated MHC I monomers, said monomers each comprising an antigenic peptide, and
streptavidin, said streptavidin bound to said biotinylated cytotoxic moiety and to said biotinylated MHC I monomers.

2. The cytotoxic MHC I conjugate of claim 1, wherein said biotinylated cytotoxic moiety is an alpha-particle-emitting radionuclide chelated to a biotinylated bifunctional moiety or is another biotinylated cytotoxin.

3. The cytotoxic MHC I conjugate of claim 2, wherein said alpha particle-emitting radionuclide is actinium-225 or bismuth-213.

4. The cytotoxic MHC I conjugate of claim 2, wherein said cytotoxin is saporin, ricin, gelonin or calicheamicin.

5. The cytotoxic MHC I conjugate of claim 2, wherein said bifunctional moiety is 1,4,7,10-tetraazacyclodododecane-1,4,7,19-tetraacetic acid or diethylenetriaminepentaacetic acid.

6. The cytotoxic MHC I conjugate of claim 1, wherein said MHC I monomers are HLA-A2 or H-2K^{d}.

7. The cytotoxic MHC I conjugate of claim 1, wherein said antigenic peptide has an amino acid sequence of one of SEQ ID NOS: 1-10.

8. The cytotoxic MHC I conjugate of claim 1, wherein the conjugate is a tetramer comprising said biotinylated cytotoxic moiety and said biotinylated antigenic peptide/MHC I monomers bound to streptavidin in a 1:4 ratio.

9. The cytotoxic MHC I conjugate of claim 8, wherein said cytotoxic moiety is said alpha particle-emitting labeled bifunctional moiety and said antigenic peptide has one of SEQ ID NOS: 1-10.

10. The cytotoxic MHC I conjugate of claim 9, wherein said alpha particle-emitting labeled bifunctional moiety is an ²²⁵Ac-labeled bifunctional moiety.

11. The cytotoxic MHC I conjugate of claim 8, wherein said cytotoxic moiety is said cytotoxin and said antigenic peptide has one of SEQ ID NOS: 1-10.

12. A method of killing a CD8⁺ T cell clonal population comprising:
contacting said clonal T cells with an effective amount of the cytotoxic MHC I conjugate of claim 1.

13. The method of claim 12, wherein said clonal T cells are contacted in vitro, in vivo or ex vivo.

14. The method of claim 12, wherein killing said CD8+ T cell clonal population selectively blocks a CD8+ T cell clone mediated disease process.

15. The method of claim 14, wherein said CD8+ T cell clone mediated disease is an autoimmune disease, graft versus host diseases or transplant rejection.

16. A method of purging a CD8⁺ T cell clonal population from bone marrow for a bone marrow transplant comprising:
contacting said clonal T cells in the bone marrow ex vivo with an effective amount of the cytoxic MHC I conjugate of claim 1; and.
transplanting the bone marrow purged of said clonal T cells into a bone marrow recipient.

17. A method of constructing a cytotoxic MHC I conjugate, comprising:
adding streptavidin to bind an admixture comprising:
said biotinylated cytotoxic moiety of claim 1; and
said biotinylated MHC I monomers of claim 1, thereby constructing the cytotoxic MHC I conjugate.

18. The method of claim 17, wherein said admixture comprises said biotinylated cytotoxic moiety and said biotinylated MHC I monomers in a ratio of about 1:3.

19. The method of claim 17, wherein said strepavidin is added to the admixture in an amount up to a 1:4 ratio.

20. A method of constructing a cytotoxic MHC I conjugate, comprising:
adding streptavidin to bind an admixture comprising:
a biotinylated bifunctional moiety or said biotinylated cytotoxin of claim 1; and
said biotinylated MHC I monomers of claim 1; and
chelating an alpha-particle emitting radionuclide to said bound biotinylated bifunctional moiety, thereby constructing the cytotoxic MHC I conjugate.

21. The method of claim 20, wherein said admixture comprises said biotinylated cytotoxic agent or said biotinylated bifunctional moiety and said biotinylated MHC I monomers in a ratio of about 1:3.

22. The method of claim 20, wherein said strepavidin is added to the admixture in an amount up to a 1:4 ratio.

23. The method of claim 20, wherein said bifunctional moiety is 1,4,7,10-tetraazacyclodododecane-1,4,7,19-tetraacetic acid or diethylenetriaminepentaacetic acid.

24. The method of claim 20, wherein said alpha particle-emitting radionuclide is actinium-225, astatine-211 or bismuth-213.

25. A cytotoxic MHC I conjugate, comprising:
biotinylated MHC I monomers, said monomers further comprising an antigenic peptide;
an alpha-particle-emitting radionuclide chelated to a bifunctional moiety, said bifunctional moiety bound to said antigenic peptide; and
streptavidin bound to said biotinylated MHC I monomers.

26. The cytotoxic MHC I conjugate of claim 25, wherein said alpha particle-emitting radionuclide is actinium-225, astatine-211 or bismuth-213.

27. The cytotoxic MHC I conjugate of claim 25, wherein said bifunctional moiety is 1,4,7,10-tetraazacyclodododecane-1,4,7,19-tetraacetic acid or diethylenetriaminepentaacetic acid.

28. The cytotoxic MHC I conjugate of claim 25, wherein said MHC I monomers are HLA-A2 or H-2K^{d}.

29. The cytotoxic MHC I conjugate of claim 25, wherein said antigenic peptide has an amino acid sequence of one of SEQ ID NOS: 1-10.

30. The cytotoxic MHC I conjugate of claim 25, wherein the conjugate is a tetramer comprising said streptavidin bound to said biotinylated antigenic peptide/MHC I monomers in a 1:4 ratio.

31. A method of killing a CD8⁺ T cell clonal population comprising:
contacting said clonal T cells with an effective amount of the cytotoxic MHC I conjugate of claim 25.

32. The method of claim 31, wherein said clonal T cells are contacted in vitro, in vivo or ex vivo.

33. The method of claim 31, wherein killing said CD8+ T cell clonal population selectively blocks a CD8+ T cell clone mediated disease process.

34. The method of claim 33, wherein said CD8+ T cell clone mediated disease is an autoimmune disease, graft versus host diseases or transplant rejection.

35. A method of purging a CD8⁺ T cell clonal population from bone marrow for a bone marrow transplant comprising:
contacting said clonal T cells in the bone marrow ex vivo with an effective amount of the cytoxic MHC I conjugate of claim 25; and.
transplanting the bone marrow purged of said clonal T cells into a bone marrow recipient.

36. A method of constructing a cytotoxic MHC I conjugate, comprising:
adding streptavidin to bind said biotinylated MHC I monomers of claim 25; and,
linking said alpha-particle-emitting labeled bifunctional moiety to said antigenic peptide of claim 25, thereby constructing the cytotoxic MHC I conjugate.

37. A cytotoxic MHC I conjugate, comprising:
a ²²⁵Ac-labeled biotinylated bifunctional moiety;
biotinylated MHC I monomers, said monomers each further comprising an antigenic peptide attached thereto; and
streptavidin, said streptavidin bound to said ²⁵⁵Ac-labeled biotinylated bifunctional moiety and said biotinylated MHC I monomers.

38. The cytotoxic MHC I conjugate of claim 37, wherein said bifunctional moiety is 1,4,7,10-tetraazacyclodododecane-1,4,7,19-tetraacetic acid or diethylenetriaminepentaacetic acid.

39. The cytotoxic MHC I conjugate of claim 37, wherein said MHC I monomers are HLA-A2 or H-2K^{d}.

40. The cytotoxic MHC I conjugate of claim 37, wherein said antigenic peptide has an amino acid sequence of one of SEQ ID NOS: 1-10.

41. The cytotoxic MHC I conjugate of claim 37, wherein the conjugate is a tetramer comprising said ²²⁵Ac-labeled biotinylated bifunctional moiety and said biotinylated antigenic peptide/MHC I monomers bound to streptavidin in a 1:3 ratio.

42. A method of killing a CD8⁺ T cell clonal population comprising:
contacting said clonal T cells with an effective amount of the cytotoxic MHC I conjugate of claim 37.

43. The method of claim 42, wherein said clonal T cells are contacted in vitro, in vivo or ex vivo.

44. The method of claim 42, wherein killing said CD8+ T cell clonal population selectively blocks a CD8+ T cell clone mediated disease process.

45. The method of claim 44, wherein said CD8+ T cell clone mediated disease is an autoimmune disease, graft versus host diseases or transplant rejection.

46. A method of purging a CD8⁺ T cell clonal population from bone marrow for a bone marrow transplant comprising:
contacting said clonal T cells in the bone marrow ex vivo with an effective amount of the cytoxic MHC I conjugate of claim 37; and
transplanting the bone marrow purged of said clonal T cells into a bone marrow recipient.

47. A cytotoxic MHC I conjugate, comprising:
biotinylated MHC I monomers, said monomers further comprising an antigenic peptide;
a ²²⁵Ac-labeled bifunctional moiety, said bifunctional moiety bound to said antigenic peptide; and
streptavidin bound to said biotinylated MHC I monomers.

48. The cytotoxic MHC I conjugate of claim 47, wherein said bifunctional moiety is 1,4,7,10-tetraazacyclodododecane-1,4,7,19-tetraacetic acid or diethylenetriaminepentaacetic acid.

49. The cytotoxic MHC I conjugate of claim 47, wherein said MHC I monomers are HLA-A2 or H-2K^{d}.

50. The cytotoxic MHC I conjugate of claim 47, wherein said antigenic peptide has an amino acid sequence of one of SEQ ID NOS: 1-10.

51. The cytotoxic MHC I conjugate of claim 47, wherein the conjugate is a tetramer comprising said streptavidin bound to said biotinylated antigenic peptide/MHC I monomers in a 1:4 ratio.

52. A method of killing a CD8⁺ T cell clonal population comprising:
contacting said clonal T cells with an effective amount of the cytotoxic MHC I conjugate of claim 47.

53. The method of claim 52, wherein said clonal T cells are contacted in vitro, in vivo or ex vivo.

54. The method of claim 52, wherein killing said CD8+ T cell clonal population selectively blocks a CD8+ T cell clone mediated disease process.

55. The method of claim 54, wherein said CD8+ T cell clone mediated disease is an autoimmune disease, graft versus host diseases or transplant rejection.

56. A method of purging a CD8⁺ T cell clonal population from bone marrow for a bone marrow transplant comprising:
contacting said clonal T cells in the bone marrow ex vivo with an effective amount of the cytoxic MHC I conjugate of claim 47; and
transplanting the bone marrow purged of said clonal T cells into a bone marrow recipient.

57. A cytotoxic MHC I conjugate, comprising:
a cytotoxic moiety; and
an MHC I monomer comprising an antibody fragment, said monomer bound to or fused to said cytotoxic moiety.

58. The cytotoxic MHC I conjugate of claim 57, wherein said cytotoxic moiety is an alpha-particle-emitting radionuclide chelated to a bifunctional moiety or is a cytotoxin.

59. The cytotoxic MHC I conjugate of claim 58, wherein said alpha particle-emitting radionuclide is actinium-225, astatine-211 or bismuth-213.

60. The cytotoxic MHC I conjugate of claim 58, wherein said cytotoxin is saporin, ricin, gelonin or calicheamicin.

61. The cytotoxic MHC I conjugate of claim 58, wherein said bifunctional moiety is 1,4,7,10-tetraazacyclodododecane-1,4,7,19-tetraacetic acid or diethylenetriaminepentaacetic acid.

62. The cytotoxic MHC I conjugate of claim 57, wherein said MHC I monomers are HLA-A2 or H-2K^{d}.

63. The cytotoxic MHC I conjugate of claim 57, wherein said antibody fragment is an IgG fragment.

64. A method of killing a CD8⁺ T cell clonal population comprising:
contacting said clonal T cells with an effective amount of the cytotoxic MHC I conjugate of claim 57.

65. The method of claim 64, wherein said clonal T cells are contacted in vitro, in vivo or ex vivo.

66. The method of claim 64, wherein killing said CD8+ T cell clonal population selectively blocks a CD8+ T cell clone mediated disease process.

67. The method of claim 66, wherein said CD8+ T cell clone mediated disease is an autoimmune disease, graft versus host diseases or transplant rejection.

68. A method of purging a CD8+ T cell clonal population from bone marrow for a bone marrow transplant comprising:
contacting said clonal T cells in the bone marrow ex vivo with an effective amount of the cytoxic MHC I conjugate of claim 57; and
transplanting the bone marrow purged of said clonal T cells into a bone marrow recipient.

69. A cytotoxic MHC I conjugate, comprising:
a ²²⁵Ac-labeled bifunctional moiety; and
an MHC I monomer comprising an antibody fragment, said monomer fused to said bifunctional moiety.

70. The cytotoxic MHC I conjugate of claim 69, wherein said bifunctional moiety is 1,4,7,10-tetraazacyclodododecane-1,4,7,19-tetraacetic acid or diethylenetriaminepentaacetic acid.

71. The cytotoxic MHC I conjugate of claim 69, wherein said MHC I monomers are HLA-A2 or H-2K^{d}.

72. The cytotoxic MHC I conjugate of claim 69, wherein said antibody fragment is an IgG fragment.

73. A method of killing a CD8⁺ T cell clonal population comprising:
contacting said clonal T cells with an effective amount of the cytotoxic MHC I conjugate of claim 69.

74. The method of claim 73, wherein said clonal T cells are contacted in vitro, in vivo or ex vivo.

75. The method of claim 73, wherein killing said CD8+ T cell clonal population selectively blocks a CD8+ T cell clone mediated disease process.

76. The method of claim 75, wherein said CD8+ T cell clone mediated disease is an autoimmune disease, graft versus host diseases or transplant rejection.

77. A method of purging a CD8⁺ T cell clonal population from bone marrow for a bone marrow transplant comprising:
contacting said clonal T cells in the bone marrow ex vivo with an effective amount of the cytoxic MHC I conjugate of claim 69; and
transplanting the bone marrow purged of said clonal T cells into a recipient.
